(19) United States
(12) Patent Application Publication
(10) Pub. No.: US 2004/0106937 A1
(43) Pub. Date: Jun. 3, 2004

(54) Clamp accessory and method for an ablation instrument

(75) Inventors: Dany Berube, Milpitas, CA (US); Ketan Shroff, Pleasanton, CA (US); Pierre-Antoine Chapelon, Carrieres sur Seine (FR)

Correspondence Address: Ross Carothers, AFx inc., 47929 Fremont Blvd., Fremont, CA, 94538, US

(73) Assignee: AFx, Inc., Fremont, CA

(21) Appl. No.: 10/602,372

(22) Filed: Jun. 23, 2003

Related U.S. Application Data
(60) Provisional application No. 60/390,740, filed on Jun. 21, 2002.

(51) Int. Cl. A61B 17/08

(52) U.S. Cl. 606/151; 606/167

(57) ABSTRACT

An apparatus and method is provided as an accessory to an ablation device or instrument. The accessory includes a first and second jaw members adapted to work with an existing ablation device during ablation of biological tissue. The first jaw member includes a partial lumen adapted to accept and hold an ablation device therein. The accessory further includes a clamping means operably attached to the jaw members to facilitate placement and retention of target tissue therebetween. The accessory may incorporate a transmurality assessment system to determined whether the ablation is complete. The transmurality system may actively or passively obtain tissue characteristics indicating transmurality. These transmurality systems may includes two or more electrodes which each measure at least one of conduction time, conduction distance, conduction velocity, phase angle, and impedance through at least a portion of the targeted tissue to determine the transmurality of the ablation lesion.

### RELATED APPLICATION

This application claims the benefit of provisional application Ser. No. 60/390,740, filed Jun. 21, 2002.

### BACKGROUND OF THE INVENTION

It is well documented that atrial fibrillation, either alone or as a consequence of other cardiac disease, continues to persist as the most common cardiac arrhythmia. According to recent estimates, more than two million people in the U.S. suffer from this common arrhythmia, roughly 0.15% to 1.0% of the population. Moreover, the prevalence of this cardiac disease increases with age, affecting nearly 8% to 17% of those over 60 years of age.

Atrial arrhythmia may be treated using several methods. Pharmacological treatment of atrial fibrillation, for example, is initially the preferred approach, first to maintain normal sinus rhythm, or secondly to decrease the ventricular response rate. Other forms of treatment include drug therapies, electrical cardioversion, and RF catheter ablation of selected areas determined by mapping. In the more recent past, other surgical procedures have been developed for atrial fibrillation, including left atrial isolation, transvenous catheter or cryosurgical ablation of His bundle, and the Corridor procedure, which have effectively eliminated irregular ventricular rhythm. However, these procedures have for the most part failed to restore normal cardiac hemodynamics, or alleviate the patient's vulnerability to thromboembolism because the atria are allowed to continue to fibrillate. Accordingly, a more effective surgical treatment was required to cure medically refractory atrial fibrillation of the Heart.

On the basis of electrophysiologic mapping of the atria and identification of macroreentrant circuits, a surgical approach was developed which effectively creates an electrical maze in the atrium (i.e., the MAZE procedure) and precludes the ability of the atria to fibrillate. Briefly, in the procedure commonly referred to as the MAZE III procedure, strategic atrial incisions are performed to prevent atrial reentry circuits and allow sinus impulses to activate the entire atrial myocardium, thereby preserving atrial transport function postoperatively. Since atrial fibrillation is characterized by the presence of multiple macroreentrant circuits that are fleeting in nature and can occur anywhere in the atria, it is prudent to interrupt all of the potential pathways for atrial macroreentrant circuits. These circuits, incidentally, have been identified by intraoperative mapping both experimentally and clinically in patients.

Generally, this procedure includes the excision of both atrial appendages, and the electrical isolation of the pulmonary veins. Further, strategically placed atrial incisions not only interrupt the conduction routes of the common reentrant circuits, but they also direct the sinus impulse from the sinoatrial node to the atrioventricular node along a specified route. In essence, the entire atrial myocardium, with the exception of the atrial appendages and the pulmonary veins, is electrically activated by providing for multiple blind alleys off the main conduction route between the sinoatrial node to the atrioventricular node. Atrial transport function is thus preserved postoperatively as generally set forth in the series of articles: Cox, Schuessler, Boineau, Canavan, Cain, Lindsay, Stone, Smith, Corr, Change, and D'Agostino, Jr., The Surgical Treatment Atrial Fibrillation (pts. 1-4), 101 Thorac Cardiovasc Surg., 402-426, 569-592 (1991).

While this MAZE III procedure has proven effective in treating medically refractory atrial fibrillation and associated detrimental sequelae, this operational procedure is traumatic to the patient since this is an open-heart procedure and substantial incisions are introduced into the interior chambers of the Heart. Consequently, other techniques have been developed to interrupt atrial fibrillation restore sinus rhythm. One such technique is strategic ablation of the atrial tissues and lesion formation through tissue ablation instruments.

Most approved tissue ablation systems now utilize radio frequency (RF) energy as the ablating energy source. Accordingly, a variety of RF based catheters and power supplies are currently available to electrophysiologists. However, radio frequency energy has several limitations including the rapid dissipation of energy in surface tissues resulting in shallow “burns” and failure to access deeper arrhythmic tissues. Another limitation of RF ablation catheters is the risk of clot formation on the energy emitting electrodes. Such clots have an associated danger of causing potentially lethal strokes in the event that a clot is dislodged from the catheter. It is also very difficult to create continuous long lesions with RF ablation instruments.

As such, instruments which utilize other energy sources as the ablation energy source, for example in the microwave frequency range, are currently being developed. Microwave frequency energy, for example, has long been recognized as an effective energy source for heating biological tissues and has seen use in such hyperthermia applications as cancer treatment and preheating of blood prior to infusions. Accordingly, in view of the drawbacks of the traditional catheter ablation techniques, there has recently been a great deal of interest in using microwave energy as an ablation energy source. The advantage of microwave energy is that it is much easier to control and safer than direct current applications and it is capable of generating substantially larger and longer lesions than RF catheters, which greatly simplifies the actual ablation procedures. Such microwave ablation systems are described in the U.S. Pat. No. 4,641,649 to Walinsky; U.S. Pat. No. 5,246,438 to Langberg; U.S. Pat. No. 5,405,346 to Grundy, et al.; and U.S. Pat. No. 5,314,466 to Stern, et al, each of which is incorporated herein by reference.

Regardless of the energy source applied to ablate the arrhythmic tissues, these strategically placed lesions must electrically sever the targeted conduction paths. Thus, not only must the lesion be properly placed and sufficiently long, it must also be sufficiently deep to prevent the electrical impulses from traversing the lesion. Ablation lesions of insufficient depth may enable currents to pass over or under the lesion, and thus be incapable of disrupting the reentry circuits. In most cases, accordingly, it is desirable for the ablation lesion to be transmural.

To effectively disrupt electrical conduction through the cardiac tissue and gap junctions the tissue temperature must reach a threshold where irreversible cellular damage occurs. The temperature at the margin between viable and nonviable tissue has been demonstrated to be about 48° C. to about 50° C. Haines et al. (Haines D E, Watson D D, Tissue Heating During Radiofrequency Catheter Ablation: A Thermodynamic Model and Observations in Isolated Perfused and Superfused Canine Right Ventricular Free Wall, PACINT CLIN ELECTROPHYSIOL, June 1989, 12(6), pp. 962-76.) Thus, to ensure ablation, the tissue temperature should exceed this margin.

This, however, is often difficult to perform and/or assess since the cardiac tissue thickness varies with location and, further, varies from one individual to another.

Most tissue ablation instruments typically ablate tissue through the application of thermal energy directed toward a targeted biological tissue, in most cases the surface of the biological tissue. As the targeted surface of the biological tissue heats, for example, the ablation lesion propagates from the targeted surface toward an opposed second surface of the tissue. Excessive thermal energy at the interface between the tissue and the ablation head, on the other hand, is detrimental as well. For example, particularly with RF energy applications, temperatures above about 100° C. can cause coagulation at the RF tip. Moreover, the tissue may adhere to the tip, resulting in tearing at the ablation site upon removal of the ablation instrument, or immediate or subsequent perforation may occur. Thin walled tissues are particularly susceptible.

Generally, if the parameters of the ablation instrument and energy output are held constant, the lesion size and depth should be directly proportional to the interface temperature and the time of ablation. However, the lag in thermal conduction of the tissue is a function of the tissue composition, the tissue depth and the temperature differential. Since these variables may change constantly during the ablation procedure, and without overheating the tissues at the interface, it is often difficult to estimate the interface temperature and time of ablation to effect a proper transmural ablation, especially with deeper arrhythmic tissues.

Several attempts have been made to assess the completion or transmurality of an ablation lesion. The effective disruption of the electrical conduction of the tissue does of course affect the electrical characteristics of the biological tissue. Thus, some devices and techniques have been developed which attempt to measure at least one of the electrical properties, such as those based upon a function of impedance (e.g., its value, the change in value, or the rate of change in value) of the ablated tissue, to determine whether the ablation is transmural and complete.

While these recent applications have been successful in part, they depend directly on the effective measurement of the electrical properties of the targeted tissue. This can be problematic when the ablation instrument cannot be held stationary with respect to the target tissue. Additionally, dynamic changes to the properties of the tissue being ablated can impact the ability to effectively measure the electrical properties of the target tissue. For example, as the ablation process commences, cellular structures breakdown. Initially, as the cellular membranes rupture during ablation, fluids are released which enhance certain electrical properties of the ablated tissue, impedance for example. As the tissue is further heated the fluids dissipate and the impedance dramatically increases. This can be problematic when the target tissue is thicker since the tissue proximate to the ablation device is farther along in the ablation process while the cellular structure near the tissue surface opposite the ablation site is just starting to breakdown. One advantage of microwave energy, over other modalities such as RF, resistive heating and Cryogenic based systems, is that microwave energy penetrates the tissue, minimizing the effects of thermal conduction.

Typical of devices which measure tissue characteristics to determine transmurality include U.S. Pat. No. 6,322,558 to Taylor et al. and U.S. Pat. No. 5,403,312 to Yates et al.; U.S. patent application Ser. No. 09/747,609 to Hooven; and WIPO Pub. No. WO 01/58373 A1 to Foley et al., each of which is incorporated by reference in its entirety.

In particular, the Hooven device employs an ablation device adapted into a clamp which helps to achieve transmurality by removing undesirable fluids from the ablation zone and compressing the tissue to a much smaller thickness. This, however, is problematic to use since the clamping device is rigid and hard to position, and compression of the target tissue may result in undesirable tissue damage.

Accordingly, it would advantageous to provide a device and method to better position an ablation instrument during an ablation procedure.

It is a related object to provide a device and method which is an accessory to an existing ablation instrument.

Another object is to provide a device and method to assess the transmurality of an ablation lesion during an ablation procedure without the need of an active interface between the ablation instrument and the target tissue.

It is another object to provide a device and method of minimizing tissue damage during the ablation process.

### SUMMARY OF THE INVENTION

These objects, and others which will become apparent upon reference to the following detailed description and attached drawing figures, are achieved by the use of a clamping device which is adapted as an accessory to an ablation instrument. The present invention provides a standalone device or an accessory to a surgical ablation device or instrument useful for facilitating tissue ablation procedures of sensitive biological tissue, such as those of internal organs. In particular, the present invention is suitable, as an accessory to an ablation device or instrument, for positioning the ablation device or instrument proximate the target tissue site. The present invention is also suitable for assessing the transmurality of an ablation lesion to electrically isolate conduction paths thereof during treatment of arrhythmia.

The accessory includes two opposing jaws which cooperate to hold target tissue therebetween during use. A first jaw of the accessory is adapted to allow a portion of an ablation instrument to be placed therethrough, the portion including at least one ablation element adapted to ablate biological tissue. With tissue placed between the jaws, the jaws may be operated to apply a desired force upon the tissue, minimizing tissue damage and dislodging any fluids which may be present in the compressed tissue. Once an ablation takes place, the accessory may be repositioned as necessary to complete one or more lesions as part of a desired lesion set.

In one specific embodiment the jaws are rigid and preformed to ablate certain specific tissue locations, around a pulmonary vein for example. In another embodiment the jaws are malleable having the ability to be formed into a desired configuration prior to the ablation procedure, the desired configuration corresponding to the target tissue configuration and desired lesion set.

In another specific embodiment, the accessory includes a transmurality system which determines when a particular ablation is completed. The transmurality system may be adapted to measure the electrical characteristics of the local tissue to measure at least one of conduction time, conduction distance, conduction velocity, phase angle, and impedance through at least a portion of the targeted tissue. In another embodiment, the transmurality assessment system is based on a passive interface to the target tissue, minimizing the impact of certain misleading signals corresponding to one or tissue characteristics. Using the transmurality assessment information, audio or visual feedback may be provided to indicate the completion of an ablation lesion. In other examples, the feedback information may be applied for automatic closed-loop control of a tissue ablation instrument.

The present invention may further include a jaw activation mechanism suitable for manual clamping by a surgeon. Alternatively, the jaw activation mechanism may include mounting elements upon the jaws which are designed to cooperate with surgical instruments allowing a surgeon to apply clamping force using standard surgical instruments such as graspers, forceps, for example. Last, another embodiment includes a jaw activation mechanism which may be remotely activated for use during less invasive procedures.

In yet another aspect of the present invention, a method is provided for ablating biological tissue using an accessory adapted to include two jaws, a first jaw configured to accept an ablation device or instrument and the second jaw acting to compress the tissue therebetween. The method includes placing a portion of an ablation device into the first jaw, the portion including at least one ablation element adapted to ablate the tissue. The tissue is placed between the jaws and the jaws are brought together, reducing the overall thickness of the tissue to be ablated and removing and fluids which may exist from the ablation area. The method further includes determining the transmurality of the ablated tissue, the indication of transmurality being used to end the ablation process.

### BRIEF DESCRIPTION OF THE DRAWINGS

FIG. 1 is a perspective view of an accessory in accordance one embodiment of the present invention.

FIGS. 2A-2C are fragmentary, side elevation views of an alternative clamping mechanism of the accessory of FIG. 1.

FIGS. 3A-3B are further perspective views of the accessory of FIG. 1 depicting the placement of an ablation instrument therein.

FIG. 4 is a perspective view of an alternate embodiment of the accessory of FIG. 1.

FIGS. 5A-5D are fragmentary, cross-sectional views of another embodiment of an accessory, in accordance with the present invention.

FIG. 6A is a fragmentary, side elevation view of the accessory of FIG. 1 engaging tissue.

FIG. 6B is a perspective view, of the accessory of FIG. 1 engaging tissue proximate the left pulmonary veins of a human heart.

FIG. 6C is a perspective view of the human heart depicting exemplary lesion sets.

FIG. 7 is a perspective view of another embodiment of an accessory, in accordance with the present invention.

### DETAILED DESCRIPTION OF THE INVENTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various Figures.

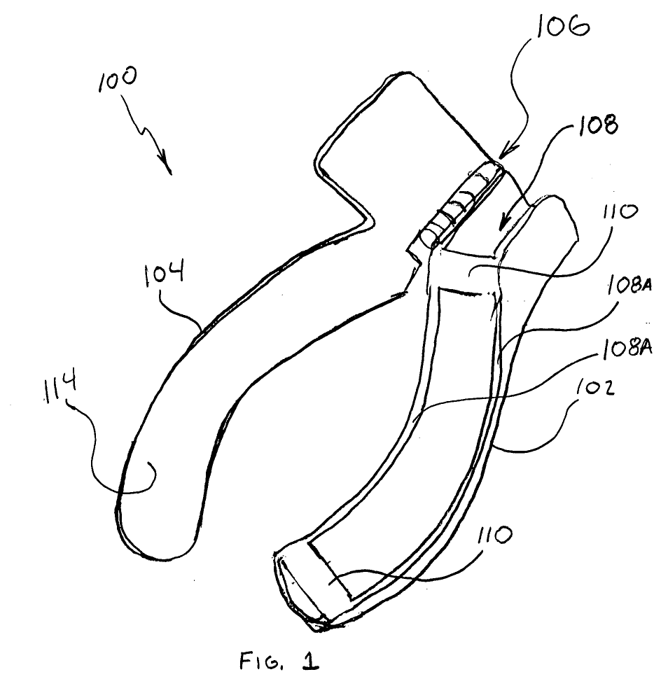

Turning now to FIG. 1, an accessory 100 in accordance with one embodiment of the present invention is provided. Accessory 100 includes two clamshell jaw portions 102, 104. Jaw 102 is adapted to cooperate with an ablation device or instrument, holding the ablation device or instrument in a desired orientation and position during an ablation procedure. Jaw 102 includes a partial lumen or groove 108 including an inner surface formed to accept the ablation device, the inner surface substantially conforming to the outer surface of the device, as is discussed in greater detail below. Jaw 102 further includes one or more cross members 110 which act to retain the ablation device once the device has been advanced along partial lumen 108.

Jaw 102 can be made from any biocompatible suitable material, such as biocompatible plastics, metals and other flexible biocompatible materials including, but not limited to, PU Pellethane, Teflon or polyethylent, which is capable of shape retention once external forces are removed. The material selected, of course, must be compatible with the ablative energy used. Jaw 102 may also include thermally isolating portions (not shown) which correspond to active ablating elements, electrodes designed to transmit RF energy ablative energy to the target tissue for example. These thermally isolating portions would act to thermally isolate the ablating device from surrounding tissue, preventing undesirable damage thereto. The thermally isolating portions may also act to protect the jaw 102 itself from the extreme temperatures associated with certain ablative modalities.

Additionally, jaw 102 may be translucent allowing a surgeon to place an ablation device within partial lumen 108, noting its position as its being advanced therein. While Jaw 102 may be rigid or malleable, jaw 102 is preformed, or otherwise shaped, to achieve a desired ablation. As shown in FIG. 1 Jaw 102 is preferably curvilinear to better grasp certain tissue for ablation, epicardial tissue of the heart for example. If malleable, jaw 102 may be manipulated to more easily engage the target tissue wherein the ablation lesion will be created.

Jaw 104 comprises an inner surface 114 which, in operation, acts to compress the target tissue between jaw 104 and the ablation device, as is discussed below. As with jaw 102, jaw 104 may be made from any suitable biocompatible material. Jaw 104 is operably connected to jaw 102 through a clamping means 106 to allow the inner surface 114 of jaw 104 to compress target tissue against the ablation device. While clamping means 106 may provide a simple point of rotation, as depicted in FIG. 1, clamping means 106 may be adapted to better accommodate thicker tissue.

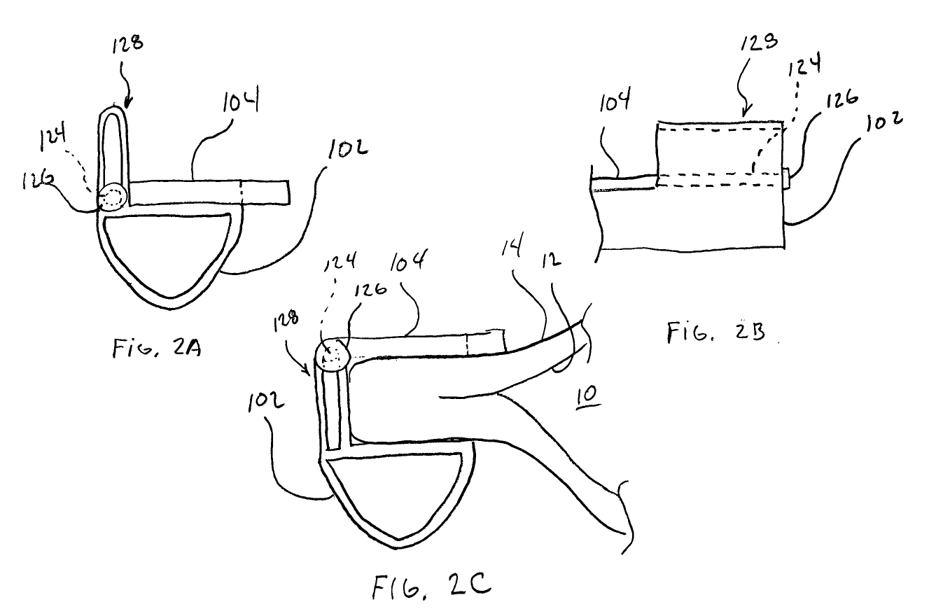

Now turning to FIG. 2, an alternative clamping means 106A will be discussed. As shown in FIGS. 2A and 2B, jaw portion 102 includes a slot portion 128. Jaw 104 includes a pin 124 which rides in slot portion 128 and is retained by end cap 126. Referring also to FIG. 2C, as a portion of heart tissue 10 is clamped within the accessory 100, pin 124 is allowed to translate within slot portion 128 to accommodate the thickness of the tissue. Additionally, it should be readily apparent that such compression will remove any fluids that may exist proximate the tissue, allowing for faster ablation to occur.

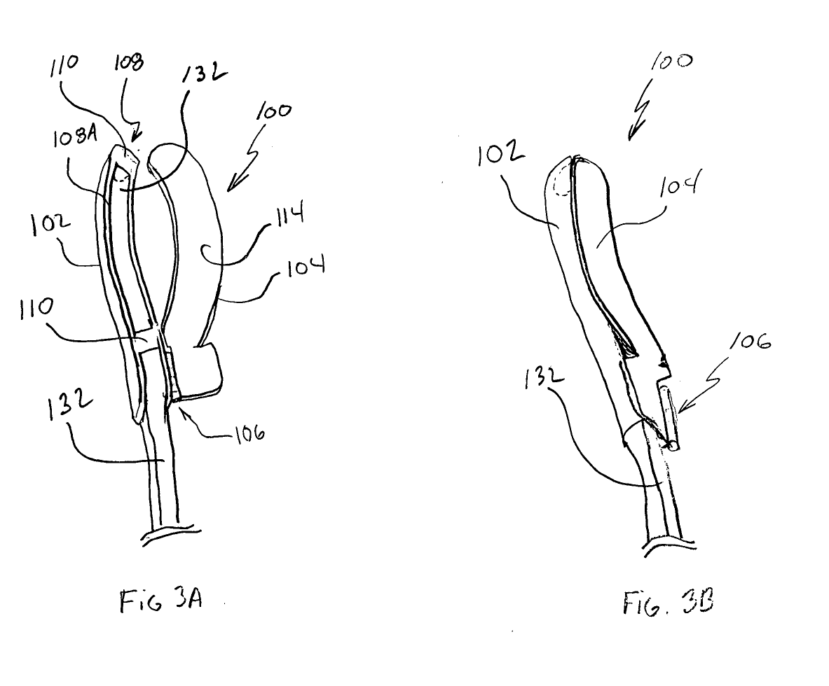

Referring now to FIGS. 3A and 3B, operation of the accessory 100 will be discussed in greater detail. As shown specifically in FIG. 3A, an ablation device 132 is advanced into partial lumen 108 until at least one ablating element is within the partial lumen 108. The ablation device 132 may be any suitable ablation device used to ablate biological tissues with ablative energy, including but not limited to, RF, cryogenic, microwave, resistive heating, chemical and light at suitable wavelengths to ablate tissue. For exemplary purposes only, one such suitable ablation device is the FLEX 10™ product manufactured and sold by AFx inc. of Fremont, Calif.

Once the ablation device 132 is properly positioned, the clamping means is activated and inner surface of jaw 104 is placed proximate to the ablation device 132, as better depicted in FIG. 3B, clamping the target tissue therebetween. The target tissue between jaw members 102, 104 is then ablated. Once ablation is complete the accessory 100, along with the ablation device 132, may be moved to another desirable location to form an additional ablation, which may or may not be continuous with the previously created lesion. Alternatively, the ablation device 132 may be advanced within the partial lumen 108 while the tissue remains clamped between the jaw members 102, 104. The tissue can then be ablated with the ablation device 132 at the new location, creating a second lesion which may or may not be continuous with the previously created lesion.

One important feature of the accessory 100 is that complete compression of the tissue between the jaw members 102, 104 is not necessary to create a lesion in the target tissue. For example, as is discussed in more detail below, if a lesion around the right pulmonary veins is desired, each jaw 102, 104 may be placed on opposite sides of the pulmonary veins, the jaws being closed a sufficient amount to hold the ablation device in place during ablation, but not enough to cause damage to the tissue or to prohibit blood flow through the pulmonary veins. While the lesion will only be created proximate the ablation device due to the cooling of the area via blood flowing in the pulmonary veins and atrium, the ablation is completed without undesirable tissue damage or reduced blood flow in the pulmonary veins which may result in further injury to the patient.

As stated above, the accessory may incorporate transmurality systems to determine when a given ablation is compete, e.g. an ablation lesion is formed throughout the depth of the target tissue. Such transmurality systems include those described in co-pending patent application Ser. No. 10/370,179, entitled, “Apparatus and Method For Assessing Tissue ablation Transmurality”, and co-pending patent application Ser. No. 10/369,887, entitled, “Apparatus and Method For Assessing Transmurality of a Tissue Ablation”, both of which are incorporated herein by reference in their entirety.

For example, with reference back to FIG. 1, the side top surfaces 108A of jaw 102 may each comprise a long electrode extending along the length of each surface 108A (not shown), the electrodes being configured to be substantially parallel to each other and positioned such that the desired ablation will be formed between the electrodes. Alternatively, a plurality of electrodes can be placed on each side top surface 108A. In any case, each electrode is adapted to selectively transmit or receive electrical signals to measure at least one of conduction time, conduction distance, conduction velocity, phase angle, and impedance through at least a portion of the targeted biological tissue, to determine the transmurality of an ablation lesion created therein. For example, during the ablation process, the impedance between two or more electrodes may be monitored, as is well know in the art, to provide an indication when the ablation lesion has been created.

Alternatively, a needle member comprising two or more ring electrodes may be adapted to either jaw 102, 104 such that the needle pierces the target tissue during an ablation process. In this way the ablation process can be monitored by collecting and interpreting tissue characteristics from the electrodes through the ablation lesion itself. Each ring electrode, as discussed in the example above, is adapted to selectively transmit or receive electrical signals to measure at least one of conduction time, conduction distance, conduction velocity, phase angle, and impedance through at least a portion of the targeted biological tissue, to determine the transmurality of an ablation lesion created therein.

It also may be advantageous under certain conditions to determine transmurality without measuring the electrical tissue characteristics of the target tissue. Such a passive measuring system is advantageous since electrical signals may provide a false indication of transmurality due to cellular breakdown during the ablation process.

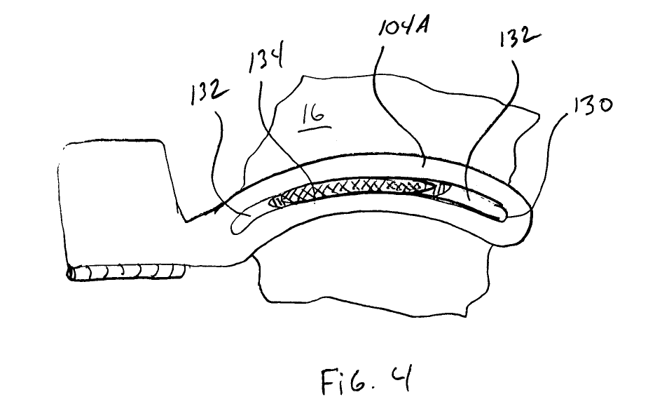

With reference now made to FIG. 4, a transmurality system which is not dependent on electrically interfacing with the target tissue is shown. Jaw 104a includes a slotted window portion 130. The inner surface of jaw 104a is then formed by a thin layer of liquid crystal sheet 132, such as that manufactured by Hallcrest of Glenview, Ill. The liquid crystal sheet 132 is adapted to change color in response to temperature. More specifically, the liquid crystal sheet 132 is adapted to provide a color change when the temperature of the target tissue at the location of sheet 132 is sufficient to cause ablation, approximately 50° C. for example. Typically the sheet 132 provides a transition of a plurality of color changes until a color denoting 50° C. is achieved. In this way, a tissue gradient is produced which provides an accurate depiction of lesion progression through the target tissue. Since the inner surface of jaw 104a is proximate the target tissue and opposite the ablation device 132, when the color change is noted, transmurality is assured.

As depicted in FIG. 4, biological tissue 16 is between jaw members 104a and 102 (opposite to jaw 104a and, thus, not specifically shown). The biological tissue 16 is being ablated and a color gradient 134 is viewable from within the window portion 130, the inner most pattern/color indicating at least a temperature in excess of what is needed for tissue ablation and transmurality of the created ablation lesion through the tissue 16 at that location. More specifically, as the temperature at sheet 132 increases proximate the ablation, the corresponding area, in response to the increased temperature, changes color. As the liquid crystal sheet 132 heats, the sheet 132 changes color until a final color is reached indicative of the desired temperature. It is important to note that once the first ablation is created the ablation device can be moved, as discussed elsewhere herein, and a second lesion, adjacent the first, can be created, the liquid crystal sheet providing visual feedback of the formed continuous lesion.

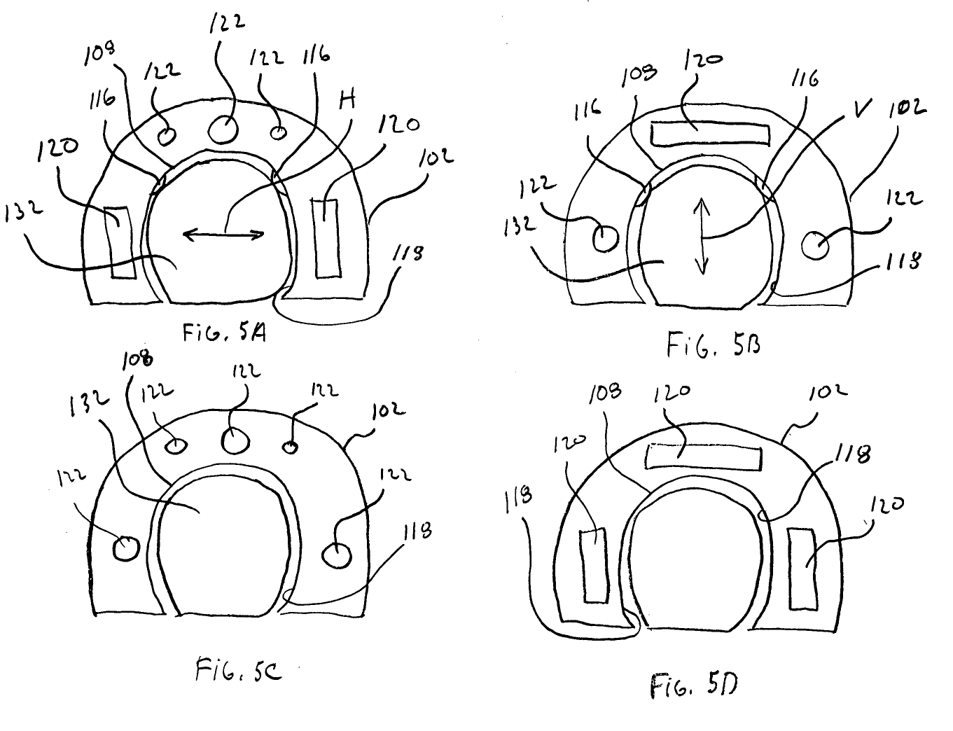

With reference now made to FIGS. 5A-5D, systems to modify the degree of malleability of the accessory 100 will be discussed in greater detail. FIGS. 5A-5D are exemplary cross-sectional views of jaw 102 comprising various elements which define the malleability of jaw 102. As depicted in FIG. 5A, jaw 102 may comprise two beam bending elements 120 which encourage bending of jaw 102 in the direction indicated by arrows H. Jaw 102 may also comprise one or more cylindrical bending elements 122 that allow for bending in all directions and offer better retention of the desired form after manipulation. Bending elements 120, 122 may be made from any suitable material having the qualities described herein.

As should be readily understood, bending elements 120, 122 may be made from materials have differing levels of malleability, such that some flexibility during compression of the target tissue can be achieved if desired. Alternatively, bending elements 120, 122 may be made from materials which, although they can be formed by hand, substantially retain their shape during use.

Still, bending elements 120, 122 may comprise materials which have differing bending characteristics along its length, allowing some areas of accessory 100 to be more malleable than others. Additionally, as will be apparent from the discussion herein, different bending elements 120, 122 may be placed along the length of jaw 102, defining the bending characteristics along it length, allowing the distal end of jaw 102 to flex while the remaining portion of jaw 102 substantially retains its shape for example. Last, if desired jaw 102 may be made from a rigid material to prevent manipulation of its form.

In the exemplary cross-sectional view depicted in FIG. 5B, accessory 100 includes a beam bending element 120 and cylindrical bending elements 122 are provided to encourage bending of jaw 102 in the direction indicated by arrows V. FIG. 5C depicts a configuration which allows for manipulation of the jaw 102 in all directions, while FIG. 5D depicts a configuration which substantially limits manipulation of jaw 102.

While the walls of the partial lumen 108 may be straight, the inner surface of partial lumen 108 alternatively may be curved, as shown in FIGS. 5A-5D. The curved nature of the walls 118 of partial lumen 108 helps to retain the ablation device therein. Additionally, one or more protrusions 116 may be formed on the inner surface of partial lumen 108, as shown, to further help position and hold the ablation device in position adjacent target tissue.

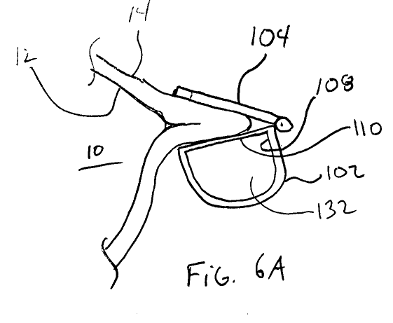

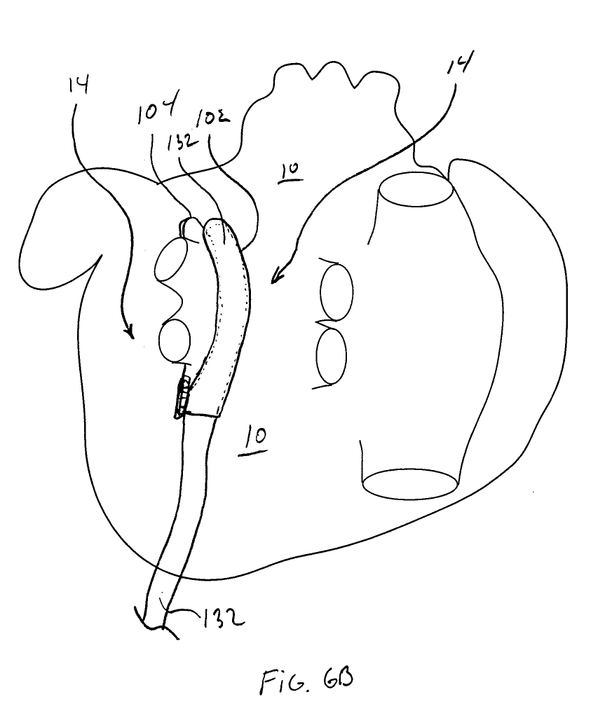

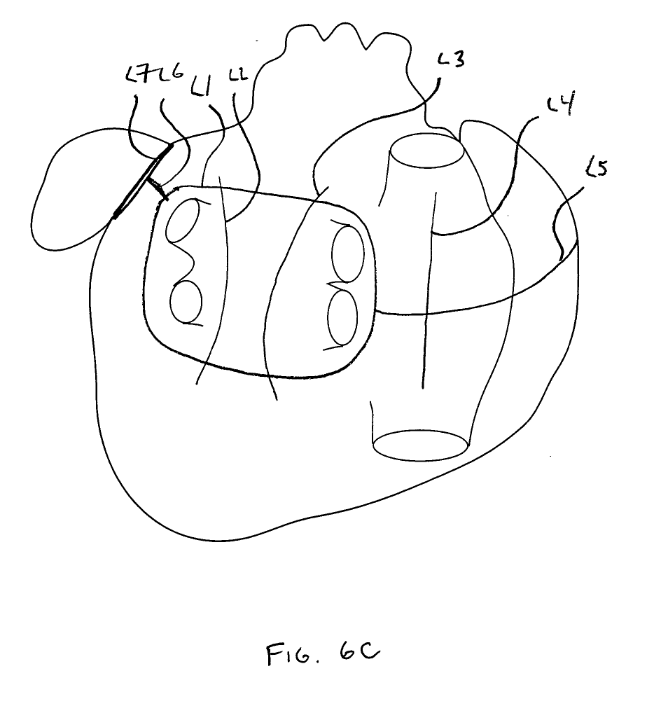

With reference now to FIGS. 6A-6C, operation of the accessory 100 will be discussed in more detail. Since the present invention is an accessory utilized with existing ablation devices or instruments, one such ablation device or instrument is generally all that is needed to complete a desired lesion set, at a tremendous cost savings and minimizing the surgeons load since the surgeon only needs to deal with the operation of one ablation device. With reference to FIG. 6A, heart tissue 10 of the left atrium is shown. A portion of the left atrium wall, having an epicardial surface 14 and an endocardial surface 12, is placed within the jaws 102, 104 of the accessory 100. An ablation tool is placed within the partial lumen 108 of jaw 102 and advanced to a point adjacent to the atrium wall wherein the desired ablation lesion will be created. Once the atrial tissue is compressed and the blood is displaced, the ablation is made. If desired, as discussed above, a transmurality system may be utilized to determine when the ablation is complete. Alternatively, the ablation may be completed by applying a predetermined amount of ablative power to the tissue for a predetermined amount of time, for the given tissue thickness. For example, it has been determined that 65 watts of microwave energy directed to cardiac tissue having a thickness of from about 3 mm to about 5 mm for a period of 35 seconds is sufficient to create an ablation lesion therethrough.

With reference now to FIG. 6B, an alternative ablation method will be discussed. Unlike folding the heart tissue 10 within the jaws 102, 104 as depicted in FIG. 6A, the jaws 102, 104 in FIG. 6B are placed around the left pulmonary veins. Once positioned, as stated above, the ablation device 132 may be advanced such that at least one ablation element is adjacent the target tissue, the atrial tissue proximate the pulmonary vein openings. The tissue is then compressed a sufficient amount to allow ablation of the target tissue from epicardial surface 14 of the heart tissue 10. It should be noted that the tissue need not be completely compressed such that the opposing walls come into contact across the surface of jaw 102 and the ablation device 132. If the blood flow through the pulmonary veins is sufficient to reduce the heating effect of the ablation device used, the ablation around the left pulmonary veins can be completed in a multiple step procedure, ablating the left side of the veins and then the right side for example, resulting in an ablation line created around the left pulmonary veins.

As stated above, the accessory 100 may not be needed for the complete set of lesions desired. However, the accessory 100 is advantageous for lesions around or proximate to the pulmonary veins, utilizing the same ablation device which is used to complete the full lesion set, saving cost and time and requiring less training by the surgeon. Exemplary lesions L1 through L7, as part of a lesion set for example, are shown in FIG. 6C. Removal of the left and/or right atrial appendages may also be part of the ablation procedure.

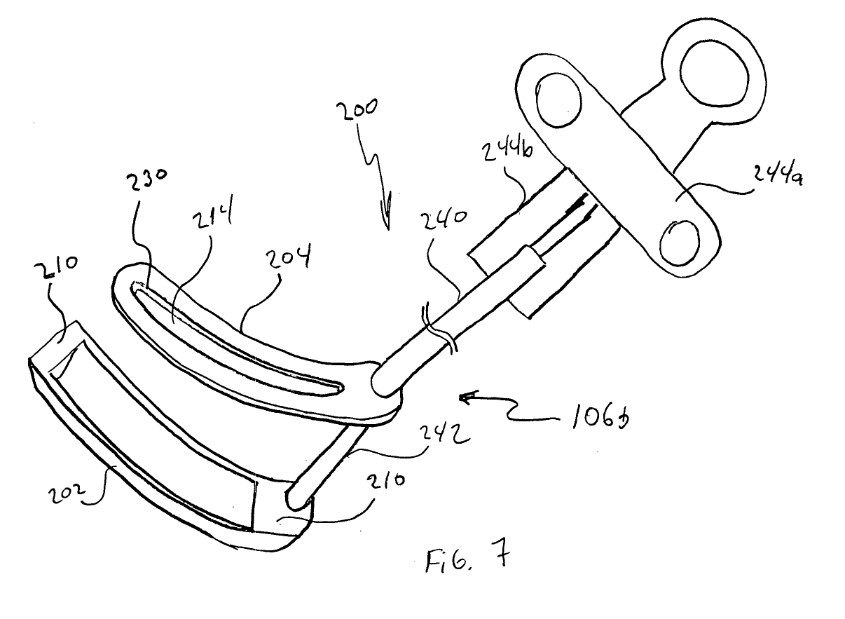

Now turning to FIG. 7, an alternative embodiment of the accessory will be discussed in greater detail. Accessory 200 is similar to accessory 100, having jaws 202 and 204. Accessory 200, however, includes a clamping means 106b. Clamping means 106b includes a first and a second tubular member, 240 and 242 respectively, and a handle 244 comprising a first portion 244a which is operably attached and translates with respect to a second portion 244b. The first tubular member 240 has a distal end which terminates at jaw 204 and a proximal end attached to second portion 244b of handle 244, as shown. Second tubular member 242 translates within tubular member 240 and has a distal end which terminates at jaw 202 and a proximal end which is attached to first portion 244a of handle 244, as shown. Thus, as handle portion 244a translates with respect to handle portion 244b, jaw 202 is advanced toward jaw 204, compressing tissue therebetween during operation. In other respects, operation of accessory 200 is similar to that of accessory 100.

Accessory 200 may also include a means for determining the distance between jaw 202 and jaw 204. The distance between jaw members 202,204 is directly related to the required power to be used during the ablation process. The distance measuring means may include visual markings located on first and second tubular members 240, 242. Alternatively, the distance can be measured electrically through the use of a linear potentiometer. For example, the body of the potentiometer can be attached to one tubular member while the pull arm of the potentiometer can be attached to the other tubular member. Therefore, as one tubular member moves relative to the other, the change in resistance of the potentiometer can be measured, such measurement being directly proportional to the distance between jaw members 240 and 242.

Furthermore, as with accessory 100, accessory 200 may includes a transmurality system as discussed herein with respect to accessory 100. As an example, jaw 204 may include a slotted portion 230 and a thin layer of liquid crystal sheet 214, as shown, allowing for a visual indication of transmurality, as discussed above.

While the present invention has been primarily described with respect to tissue ablations of the heart, it will be appreciated that the ablation systems disclosed herein may just as easily be applied to ablation of other soft tissues of the body.

Although the foregoing invention has been described in some detail for purposes of clarity of understanding, it will be apparent that certain changes and modifications may be practiced within the scope of the appended claims.

What we claim is:

1. A clamp accessory for an ablation device, comprising:
a first jaw member having a partial lumen adapted to receive the ablation device therein;
a second jaw member opposed to the first jaw member; and
a clamping means for operably attaching the first jaw member to the second jaw member, whereby operation of the clamping means results in closure of the first and second jaw members upon a target tissue.

2. The accessory of claim 1, wherein the first jaw member includes at least one cross member for retaining the ablation device therein.

3. The accessory of claim 1, wherein the first jaw member includes at least one thermally isolating portion, whereby tissue adjacent the first jaw member is protected from undesirable damage during an ablation procedure.

4. The accessory of claim 2, wherein the first jaw member comprises an inner surface adapted to receive the ablation device therein.

5. The accessory of claim 4, wherein the first jaw member further comprises at least one protrusion upon an inner surface of the partial lumen.

6. The accessory of claim 1, further comprising a transmurality system, whereby the completion of an ablation lesion can be determined.

7. The accessory of claim 6, wherein the transmurality system comprises at least two electrodes adapted to selectively transmit or receive electrical signals to measure at least one of conduction time, conduction distance, conduction velocity, phase angle, and impedance through at least a portion of the targeted biological tissue, whereby the transmurality of an ablation lesion is determined.

8. The accessory of claim 7, wherein the partial lumen defines a first and a second top side surface and a first and a second of the at least two electrodes are mounted upon the first and the second top side surface, respectively.

9. The accessory of claim 6, wherein the transmurality system is passive.

10. The accessory of claim 9, wherein the transmurality system comprises a liquid crystal sheet adapted to provide a color change in response when a desired ablation temperature is reached, the liquid crystal sheet mounted to and forming an inner surface of the second jaw.

11. The accessory of claim 10, wherein the liquid crystal sheet is adapted to change color when a temperature from about 48 C to about 52 C is reached.

12. The accessory of claim 11, wherein the liquid crystal sheet is adapted to provide a color gradient corresponding in response to a temperature gradient observed.

13. A method of ablation a target tissue with an ablation device, comprising the steps of:
providing an accessory, comprising:
a first and a second jaw member, the first jaw member having a partial lumen therein adapted to accept the ablation device; and
a clamping means for operably connecting the first and second jaw members;
positioning the ablation device within the partial lumen of the accessory;
positioning the target tissue between the first and second jaw members; and
applying energy to the ablation device to form a first ablation lesion in the target tissue.

14. The method of claim 13, wherein the step of providing an accessory comprises the step of providing a means for determining the transmurality of an ablation lesion.
